(19) United States
(12) Patent Application Publication
(10) Pub. No.: US 2020/0345970 A1
(43) Pub. Date: Nov. 5, 2020

(54) MULTIMODAL, MODULAR, MAGNETICALLY COUPLED TRANSCUTANEOUS AURICULAR STIMULATION SYSTEM INCLUDING APPARATUS AND METHODS FOR THE OPTIMIZATION OF STIMULATION AND THERAPEUTIC INTERVENTIONS

(71) Applicants: Thomas Anthony La Rovere, Santa Ynez, CA (US); Jonathon M. Honeycutt, St. Petersburg, FL (US)

(72) Inventors: Thomas Anthony La Rovere, Santa Ynez, CA (US); Jonathon M. Honeycutt, St. Petersburg, FL (US)

(21) Appl. No.: 16/863,936

(22) Filed: May 4, 2020

Related U.S. Application Data
(62) Division of application No. 62/843,446, filed on May 4, 2019.

(51) Int. Cl. A61M 21/02; A61N 1/36; A61N 5/06; A61H 21/00; A61H 23/00

(52) U.S. Cl. CPC A61M 21/02; A61M 2021/0022; A61N 1/36036; A61N 5/0622; A61H 21/00; A61H 23/00; A61N 2005/0653; A61N 2005/0652; A61N 2005/0647; A61H 2201/5007; A61H 2201/5046; A61M 2230/65; A61M 2230/08; A61M 2205/3306; A61M 2230/06; A61N 1/36034

(57) ABSTRACT

An improved modular, multi-modal neurostimulation system, includes signal generating, conditioning, and control electronics, stimulation monitoring electronics, and wearable, magnetically coupled energy emitter modules configured for coupling energy emitters to the ear for transcutaneous energy delivery to cranial nerves neurologically accessible in the auricular nerve field. Three energy modalities are available via electrodes for electrical stimulation; optical emitters for electromagnetic stimulation; vibrational emitters for vibrational stimulation. Energy modalities are selectable singularly or in combination. Stimulation control electronics may be integrated within an ear-worn coupling body or located in an external computerized device. Integrated bodily status and biofeedback sensors work with a programmable algorithm to enable closed loop operation based on monitored biofeedback activity, bodily activity and indicia of autonomic functioning. A method for optimizing stimulation using cardiorespiratory feedback and monitoring of divisional indicia of autonomic nervous system function is discussed. Disclosed is a method for optimizing therapeutic interventions via neurostimulation and a method for self-administered neurostimulation and training users in self-administered neurostimulation. Also disclosed is a stimulation coupling apparatus comprising a contour conforming plastic composition for coupling stimulation energy emitters to the auricle of a human being.

### CROSS-REFERENCE TO RELATED APPLICATIONS

This application claims priority of Provisional Application No. U.S. 62,843,446 filed on May 4, 2019 by inventors Thomas A. La Rovere and Jonathan M. Honeycutt

### BACKGROUND

Research in the field of transcutaneous neural stimulation has yielded key insights into the neurological pathways available through energy stimulation of the cranial nerves accessible in the via auricular nerve field. Improvements included in the present invention provide a flexible, modular, multimodal neurostimulation platform for furthering research and developing advanced therapeutics, with gains in efficacy, neurological interfacing, and integration with biofeedback technologies and adjunct clinical methodologies.

### FIELDS OF INVENTION

The present invention includes apparatus and methods belonging to the fields of neurostimulation and neurostimulation-enhanced interventional therapeutics for health, healing and wellness. Neurostimulation may be broadly defined as the application of energy, electrical, electromagnetic, and vibrational energy, to nerves targeted directly or indirectly (e.g., by applying energy to surrounding, connecting and/or conductive tissues, e.g., skin, tissues and vasculature), for the purpose of producing beneficial changes in the activity of neurotransmitters, the activity within structures and circuits of the brain, in neuronal and synaptic activity, and as streams of cascading effects producing changes in the activity of organs, particularly organs in communication with the autonomic nervous system, including the brain and all of the organ systems controlled or modulated by the vagus nerve—the primary cranial nerve target of the present technology.

Neuromodulation is unique among health, wellness and medical interventions and therapeutic modalities in that, because the intervention involves the application of modulated energy to the certain areas of the human body, its electronic delivery to a user may be controlled by a computer locally or remotely, through a computer communications network.

The present invention improves on earlier inventions seeking to maximize the coupling of essential neurostimulation conductors targeting the cranial nerves in the auricular nerve field of a human. Composing an optimal link between a neurostimulation device and the auricular nerve field of a person poses a number of critical challenges including wear-ability, comfort, discretion (or privacy), attachment security, simplicity of application, maximization of energy conduction to target nerves, etc. Each of these challenges can, when unanswered or inadequately answered, become barriers to therapeutic effectiveness, usability, and ultimately to consumer adoption, use and reliance.

Hereinafter, the terms “neuromodulation,” “neuro-stimulation,” “neurotherapy” and “nerve stimulation” are used interchangeably and refer to the full range of therapeutic modalities and methods by which energy, as electricity and, in the future, electromagnetic and vibrational energy, may be electronically delivered to anatomical structures of a mammalian body, such structures including nerves, tissues including connective tissues, organs individually and systemically, muscles, vasculature, glands; and to refer to devices designed and used to accomplish such energy delivery through direct or indirect application to said anatomic structures, using external, non-invasive, minimally-invasive and invasive means.

Invasive methods require the surgical implantation of a pulse generator and an electrode or conductive wire into the body where it is surgically attached to the cervical branch of the vagus nerve. Direct connection to the vagus nerve is advantageous for obvious reasons, the most important of which is the maintenance of conductive contact and the flow of stimulation energy. Devices designed for noninvasive (transcutaneous) stimulation of the vagus nerve have yet to achieve a similarly constant and reliable connection between stimulator and nerve target.

Invasive stimulation of the vagus nerve via stimulator implant has been successfully used for at least twenty-five years and has been approved by the FDA for the treatment of epilepsy and treatment resistant depression, and is under intensive study to treat a plethora of other conditions including anxiety, insomnia, migraine, weight loss, management of pain, obesity, Alzheimer's disease, and Parkinson's disease, among others. The advantages of using implantable stimulation devices is their constant, wired connection to the nerve, long life batteries, and their resistance to tampering by the patient (when the signal generator is implanted under the skin) by virtue of their effective inaccessibility to the patient. Disadvantages of invasive nerve stimulation devices include their cost, the expense of surgical implantation, the need for follow-on surgeries to change batteries and replace faulty or outdated nerve stimulator electronics, complications of wound care, the risks and dangers of surgery including infections, nerve damage, anesthesia risks and the patient's lack of control over the stimulator and dependence on expensive physician intervention.

Another class of invasive nerve stimulation devices includes those classified as “minimally invasive” which incorporate a needle electrode or an array of electrode needles which pierce and penetrate the skin to produce percutaneous nerve stimulation. U.S. Pat. No. 9,662,269 B2 describes a recent variant of these percutaneous nerve stimulation devices. Another example of such a system is disclosed in U.S. Patent Publication No. 2013/0150923, reflecting a device sold by Biegler GmbH under the trade name P-STIM®. A significant drawback of such systems is that the needle electrodes break the skin, causing pain and the consequent patient aversion, as well as the risk of infection. Additional disadvantages of such semi-invasive percutaneous stimulation devices include their relatively high cost, the expense of surgically implanting skin piercing percutaneous needle electrodes, the need for follow-on surgeries to re-position needle arrays, complications of wound care, the risks and dangers of surgery including infections, nerve damage, the pain of percutaneous needle puncture, and the patient's lack of control over the stimulator.

A compelling case for transcutaneous auricular nerve stimulation comes from clinical research on animals and human beings demonstrating that electrical stimulation of the auricular branch of the vagus nerve (ABVN) using ear-mounted electrodes produces activity detectable by function MRI (fMRI) in various key centers of the brain, including, inter alia, the nucleus of the solitary tract (NST) and the accumbens nucleus, also known as the “reward center” of the brain wherein synaptic activity is known to modulate various forms of stimulus and reward seeking behavior associated with eating, sex and the use of drug and alcohol. Neuromodulation of activity in the accumbens nucleus during transcutaneous electrical stimulation of the auricular branch of the vagus nerve has been demonstrated using functional MRI (fMRI).

Interest in the auricular nerve field has led to research regarding the alignment of electrode conductors with target nerves to minimize nerve-electrode distance, reduce resistance and signal intensity, and to avoid problems with tissue damage from unnecessarily high currents and signal scatter. Anatomical locations most proximal to underlying nerves were studied to determine the most ideal electrode placements. Findings from functional MRI studies indicate that cymba conchae stimulation, compared to earlobe (control) stimulation, produced significant activation of classical central vagal projections, e.g., widespread activity in the ipsilateral nucleus of the solitary tract (NST), bilateral spinal trigeminal nucleus, dorsal raphe, locus coeruleus, and contralateral parabrachial area, amygdala, and accumbens nucleus. Bilateral activation of the paracentral lobule was also observed. Deactivations were observed bilaterally in the hippocampus and hypothalamus. These findings provide strong evidence that, in humans, the central projections of the auricular branch of the vagus nerve (ABVN) are consistent with classical central vagal projections and can be accessed non-invasively via stimulation of the human auricle. Hereinafter, the terms cymba conchae, cymba concha, concha cymba, concha cymbae are used interchangeably.

Four primary sensory nerves area found in the externally projected dimensions of the human ear referred to as the auricle: the auriculotemporal nerve (ATN), a branch (v3) of the trigeminal nerve; the great auricular nerve (GAN), the auricular branch of the vagus nerve (ABVN), and the lessor occipital nerve (LON). A transcutaneous method of nerve stimulation may target one or more of these nerves in the auricular nerve field singly or in combination.

Disclosed is a modular system for auricular nerve field stimulation employing selectable energy emitter modules which may be configured as traditional electrodes delivering electrical energy, as optical emitters of electromagnetic energy emitting wavelengths in range from 400 nm to 1600 nm, and as emitters of vibrational energy. A closed-loop, biofeedback-based neurostimulation system is also disclosed along with methods for using neurostimulation to enhance the effectiveness of other therapeutic interventions, and for training users in self-administered stimulation methods.

In certain locations, the nerves within the auricular nerve field advantageously lay a mere 1 to 2 millimeters below the skin surfaces of the human auricle. Despite this close surface proximity, there are a number of challenges in designing the interface coupling the stimulation device to the human tissue overlaying selected nerve targets within the auricular nerve field. The most significant of these challenges are electrode attachment security, electrode comfort for the user, electrode resistance to de-coupling and electrode application complexity.

### PRIOR ART

Cerbomed GmbH manufactures a transcutaneous tVNS device (NEMOS), with a handheld controller connected by wire to an earpiece that wedges two metal electrodes, one anode and one cathode, against the skin of the cymba conchae of the ear at two points between 5 and 12 millimeters apart. This scaffold-like coupler earpiece retains the position of the electrodes and maintains the constant contact forces of the electrodes against the skin via spring forces created between superior and inferior anchor-points, with a lower “earbud-like” component positioned inferiorly in the cavum concha and a superior component wedged under the superior ridge of the cymba conchae. Positional retention of this ear piece relies on constant spring forces which are adjusted by the user using a sliding mechanism on the lower part of the scaffolding. As there is little subcutaneous padding in the skin about the superior and inferior contact points on the ear, the spring force required for position retention and the electrode contact maintenance may be poorly tolerated over prolonged periods of treatment. The NEMOS® scaffold electrode would be unsuitable for wearing during sleep. Additionally, this coupling scheme is limited to a single position which may be sub-optimal for many users, as the auricular nerve matrix and nerve fiber density per electrode attachment point can vary from one individual to another. Hence, the Nemos® lacks the flexibility to work effectively when alternate electrode sites may be preferred or required. Attachment of the Nemos® earpiece is maintained by anchoring it in the inferior portion of the cavum concah below the conchal bowl with a hollow circular piece of plastic that partially or completely occludes access to the ear canal. This means that the gravity drag of the cable weight and any additional gravity or sheering forces that may be suddenly applied to the cable, for example by snagging the cable on a table corner or any one of thousands of other snag-risks, or by dropping the handheld controller, are immediately transferred to the anchor sitting in the conchal bowl, and thereby to the concha and the lower ear itself, potentially resulting in pain and injury to these sensitive tissues and psychological distress. Relying on spring forces created by wedging the superior end of the scaffold-like ear-piece against the superior ridge of the cymba conchae reduces the earpiece's resistance to motion-generated displacement, vibration and sheering forces which may be encountered during ordinary activities of daily living.

A necessary condition for therapeutically effective transcutaneous nerve stimulation is a securely attached electrode that is, at the same time, easily, quickly and painlessly applied and removed by a user. Unlike percutaneous electrode coupling schemes which incorporate skin piercing needles, as described by U.S. Pat. No. 9,662,269 B2, transcutaneous electrode coupling avoids the complications and risks to the user presented by a skin piercing needle electrode or needle array, said complications including wound infection, pain to the user, and the need for professional attachment, removal and re-attachment. Contemporary transcutaneous electrode-skin couplers include the use of adhesives collars surrounding the electrode and affixing it to the skin; spring-loaded clips which clasp the pinna, auricle, concha or lobes of the ear, and the use of cavity-penetrating projections inserted into the ear canal as an anchoring scheme. Each of these transcutaneous electrode-to-skin coupling schemes present potential and actual complications and challenges for a user. The use of adhesive collars is highly problematic on an uneven surface such as the human ear and the use of adhesive to secure an electrode against ear tissue while withstanding gravity, motion and sheering forces may, upon removal, cause pain to the sensitive tissue of the ear of a user and require vigorous, skin irritating clean-up of the adhesive. Some manufacturers employ a spring-loaded, clip-based electrode attached to the ear lobe or to the tragus, concha or pinna of a user's ear. These ear-clip electrodes are attached to the stimulator device by a cable of at least twenty-five inches in length. The combined weight of the clip itself, the electrode, or electrode pair and its connecting cable necessitate the use of a clamping force sufficient to hold the clip in place against the weight of the clip and cable for the entire period of stimulation in situations where the ordinary movement of a wearer can easily cause electrode-clip detachment. The clamping force exerted against sensitive ear tissue for prolonged periods is a known source of discomfort to the user that can create a negative association in the mind of a user with stimulation therapy that may discourage compliance with a prescribed treatment regimen, especially when the clamping is accompanied by perceivable, slightly uncomfortable electrical stimulation. Cavity-anchoring electrodes inserted into the ear canal avoid the unpleasant clamp force of ear-clip electrodes but not the gravity drag of the cable. Such ear-canal electrodes also block the ear canal and tend to collect the waxy exudate present in the ear canal. The ear canal itself contains sensitive tissues and other structures that may be negatively affected by the insertion and wearing of inserted electrodes which plug the ear canal. One example of the ear-canal anchoring scheme is device made by Nervana® which uses the ear canal as both an anchoring structure for maintaining position and as a stimulation point, potentially accessible the auricular branch of the vagus nerve. The Nervana® ear-canal plug incorporates two conductive electrodes on what is essentially an audio-emitting ear-canal plug or “bud.” A drawback with this ear-canal electrode anchoring scheme is illustrated by the fact that, according to its crowd-funding website, Nervana LLC has received various complaints from users about “burning” sensations in the ear canal. Users of the Nervana device are instructed to use a saline solution for conductive coupling inside the ear canal. This results in the uncomfortable presence of conductive liquid in the ear canal, which is known to loosen and mobilize ear wax which may become attached to the inserted ear electrode. The ear canal is also associated with the Arnold Reflex, an uncomfortable coughing or gagging response triggered by the insertion of matter into the ear canal, including innocuous objects like Q-Tips.

### OBJECT AND ADVANTAGES

Currently marketed transcutaneous nerve stimulation devices produce less than optimal results for a number of reasons. The barrier of skin and tissues between the stimulation emitter (e.g., electrode) and a nerve inside the body generates strong electrical resistance which reduces the energy of the electric signal delivered to the target nerve. This resistance barrier can be mitigated by increasing electric potential at the cost of producing collateral effects such as burning the skin and causing pain to a user, as well as overstimulating the nerve and inadvertently stimulating adjacent nerves through signal scatter. Most currently marketed transcutaneous auricular neurostimulation devices do not, over time, adequately maintain a constant degree of coupler apposition to the skin, resulting in fluctuating, inconsistent and higher impedance which may reduce the degree of signal transmission through the skin, thereby reducing the strength of the signal reaching the target nerve. The security and stability of the electrode coupler are required for the positional constancy and the maintenance of conductive contact between electrode and the body of the user. Poor, inconsistent or unreliable position maintenance of the electrode-coupler may disrupt the conductive pathway to the target nerve, causing ineffective treatment.

The present invention includes the application of three types of energy stimulation modules: the first having electrodes configured for traditional transcutaneous electrostimulation;

the second having optical emitters configured for electromagnetic stimulation, a modality which takes advantage of the fact that light can be passed through the skin and its electromagnetic energy deposited in tissues including nerve fibers; and vibrational energy emitters. In the present disclosure, emitters of electric energy, electromagnetic or light energy, and vibration emitters, are referred to as “electrodes,” “energy emitters,” “emitters” and the like.

The electrostimulation module of the present invention includes two electrodes which are positioned on opposing sides of the ear, i.e., the ventrolateral (front) and dorsolateral (rear) surfaces of the auricle, forming an electrical path between said electrodes that passes through ear tissues and intersects one or more targeted nerves. In our experiments, nerve intersecting electrostimulation reduced the amount of energy required to deliver stimulation to the nerve by as much as thirty-five percent. The lower energy spend brings the intensity of electrostimulation current down below the pain threshold, reduces the likelihood of skin burns, and thereby reduces barriers to treatment compliance, namely discomfort, pain and skin burns. Additionally, recent clinical research has shown that nerve stimulation is more clinically efficacious at lower energy levels, which is consistent with Yerkeys-Dodson law of optimal arousal. Yerkeys-Dodson law describes an empirical relationship between arousal and performance wherein performance increases with physiological arousal, but only up to a point, beyond which more arousal causes lower performance. Applied to neurostimulation, Yerkeys-Dodson law describes a relationship between stimulation-induced arousal and performance as the neurological effects produced by or in response to stimulation. Yerkeys-Dodson law predicts that stimulation effects increase with stimulation-induced arousal, but only up to a point, beyond which more stimulation produces inferior or less effective clinical outcomes. The empirical relationship described by Yerkes-Dodson Law is often illustrated graphically as a bell-shaped performance curve which increases and then decreases with higher levels of arousal or, as we have described, stimulation as shown in FIG. 8. High stimulation current levels may over-arouse target nerves, the nervous system and the brain itself, thereby dampening the beneficial effects of stimulation or generating a refractory response to stimulation.

The invention presented herewith provides an integrated coupler-emitter array in a preferred embodiment as an ear-piece comprising a main dorsal body worn posteriorly behind the ear within the groove space between the external ear and the head sometimes referred to as the “fold” or the “crotch” of the ear, which hereinafter shall be used interchangeable to refer to the dorsal and dorsolateral dimensions and areas of the external ear (or “auricle”). One of the advantages of the behind-the-ear design is that the weight of the cable connecting it to the signal generator is distributed to the superior arc of the bow where it loops around the apex of the ear, thereby unloading the electrodes or “energy emitters” from potential dislodging weight of cables. A second advantage is that the behind the ear packaging can provide the space needed for the integration of stimulation, biofeedback and communication electronics. The ear-worn coupler design also provides significant protection against the sheering forces created by normal body and cable movement which can, as discussed above, exert forces that reduce consistent conductive electrode contact with the skin. The ear-piece design takes advantage of the crotch between the ear and the head and dorsally near the top of the ear, which provides a large, natural retention groove that securely anchors the ear-piece in position, even during movement of the wearer. Anchoring is further enhanced by the ear-coupler looping around from the crotch of the ear posteriorly to the ventral-ventrolateral side of the ear.

The present invention employs a dorsal body worn behind the ear with a cable conductor looping over the apex to the ventrolateral side of the ear where it connects to a pod having an electrode. Worn behind the ear, the dorsal body may have one or more energy emitters located on its forward ventral side making contact with the crotch of the auricle and additional emitters located on the distal-facing side of the dorsal body which has contact with the broader auricular nerve field across the crura of the antihelix (superior), triangular fossa, and scaphoid fossa, lower crus of the antihelix, upper crus of the helix, and navicular fossa. The pod is worn on the ventrolateral surface of the auricle positioned according to nerve target selection. Coupling is accomplished by the magnetic attraction of magnets and/or magnetically attractive elements located inside the dorsal body and the pod. Nerve intersecting electrical stimulation is accomplished by manually positioning the pod and holding it in place (usually requiring no more than 1 to 2 seconds), causing the dorsal body to move to an opposing, intersecting position by magnetic attraction. Multiple pods may be used to target multiple nerves.

Electrical microcurrent from two opposing electrodes on the front and rear (ventrolateral and dorsolateral sides) of the auricle pass directly through the intervening tissues and into nerve fibers within a small perimeter of the scatter-path of electrical current. Magnetic attraction between the rear-worn dorsal body and the front-worn pod effectively couples the device to the auricle. This magnetic coupling is remarkably resistant to movement displacement and avoids the pinch pain produced by spring-clip coupling schemes. Magnetic coupling has also proven effective for maintaining conductive connection between electrodes and auricular anatomical landmarks associated with the location of target nerves. Magnetic coupling is also effective for coupling skin-contact sensors, particularly photo-optical sensors used in photoplethysmography to monitor cardiorespiratory activity (as described herein) which can provide biofeedback indicia for the autonomic nervous system.

The electromagnetic stimulation module (or “pod”) disclosed with the present invention includes one or more optical emitters (i.e., LEDs) which may be arranged for direct transdermal stimulation or for nerve intersection when two optical emitters positioned on opposing sides of the ear, i.e., the ventral-ventrolateral and dorsal-dorsolateral surfaces of the auricle. Transcutaneous photo-optical stimulation of the vagus nerve and other auricular nerves is a nascent modality discovered by one of the inventors (Honeycutt). Electromagnetic or photo-stimulation offers unique and clinically significant advantages over electrical stimulation. Light energy passes easily through human skin and may be absorbed by targeted tissues, including nerve tissues. Light energy in the infrared band can easily penetrate skin tissue to stimulate targeted nerves in the auricular nerve field with virtually no risk of the skin burns associated with electrical electrodes.

A growing body of research suggests that vagus nerve stimulation can alter the synaptic environment, excite or inhibit synaptic action, activate and deactivate neural circuits and induce neuroplasticity to repair disrupted neurocircuits. Although much research remains to be done, it is believed that the relative neuroplasticity of key brain circuits, networks and brains centers connected to the vagus nerve can both inhibit and enhance the effects of therapeutic interventions. Through vagus nerve stimulation, a wide variety of therapies could realize improvements in effectiveness, therapeutic potency, outcome durability and resistance to retrogression and relapse. Psychological interventions involving cognitive therapy which rely on the brain's ability to process, store, recall and make, break and substitute associations could be enhanced by a more neuroplastic and hence more fluid and receptive neural environment.

Practitioners involved in manipulating the body and working with muscles have long known that injuries create neurological sets involving patterns of protective guarding, bracing, freezing and bypassing that interfere with and prolong recovery and elevate pain and discomfort. Each of these injury and/or pain protection sets involve a reorganization of the normal neural pathway for muscle recruitment and functional performance, e.g., walking, sitting, standing, bending, etc. In many cases, especially when an injury, dysfunction or illness has been either severe or prolonged, a traumatogenic neural set bypass can become a neurological barrier to recovery, like a cast that must be broken apart to enable the full restoration of function. Neuroplasticity-enhancing stimulation applied to the functional circuits of the brain and their controlling brain centers like the amygdala and Nucleus tractus solitarius (NTS), may awaken sidelined circuits previously shunted and/or shut-down by the brain responding to trauma. Stimulation signals applied to the vagus nerve may produce a kind of neurological orienting reflex or reset that shifts a gateway or a neural circuit out of shunt and back into phase.

Many therapeutic interventions are applied in a series of steps, stages and phases that include a preparatory phase, an intervention phase, and a recovery phase or repeated groups of these three (or more) phasic components or stages. Yet many therapeutic interventions that should have phasic features or components lack them not due to incomplete methodology, improper execution or inadequate practitioner training. Counter-intuitively, psychological therapies are the most common example of inadequately staged interventions. A general lack of awareness about the need for staging interventions pervades the psychology community in particular and results in higher rates of therapy failure, refractory outcomes and wasted therapy dollars and lost opportunities for healing.

Emerging empirical evidence indicates that neurostimulation protocols can be designed and used to optimize the patient's neural environment for each phase of therapeutic intervention, based on individual circumstances, patient factors, intervention factors and therapy goals. A relaxing, low stimulation frequency could be used in the preparatory, warm-up phase of an intervention. In physical therapy rehabilitative exercises aimed at improving or restoring limb, joint, hand and digit functions (e.g., after stroke or reconstructive surgery), an exercise regimen that begins with slow movement and recruitments and progresses to faster, stronger, more rotational, or more weight-bearing, or more discrete motor operations could paired with stimulation wherein the frequency (starting with a low, warm-up frequency) was raised step-wise as the therapeutic intervention progressed along a continuing of functionality. In cases of injuries or illness involving a loss of function, a first phase of neurostimulation to enhance a therapeutic intervention might begin with a mid to high frequency (or a mid to high amplitude) as a means of re-activating or reorganizing neural circuits sidelined by trauma or pathology. Because circuit reactivation may produce temporary homeostatic disruptions, an initial ramped-up stimulation frequency or amplitude surge could be followed by lower, calming stimulation frequencies and lower amplitudes.

For many therapeutic interventions, including psychological therapies, the most beneficial, receptive neurological environment may depend on the target pathology and is associated neurocircuits and targeted therapeutic objectives, including phasic targets such as neuronal inhibition and excitation; re-activation and reorganization of neurocircuits, neurotransmitter levels and so on. A primary guiding principle for this method of enhancing the effectiveness of therapeutic interventions using neurostimulation is, first identify the most beneficial neurological environment for the planned interventions or, preferably, for each phase of the intervention. A second principle is to parameterize neurostimulation to facilitate the identified most beneficial neurological environment. A third principle is to monitor the patient's biological markers to assess indicia of the patient's status relative to the therapeutic invention during each of its successive stages. A fourth principle is to responsively employ neurostimulation during the therapeutic invention to maintain the most beneficial neurological environment for each phase of the therapeutic intervention. A fifth principle is that the pulse rate or frequency and intensity of stimulation is often positively correlated with the level of neurological arousal subject to Yerkes-Dodson law of optimal arousal. A temporal or time-based method of using neurostimulation to enhance the effectiveness of therapeutic interventions is described in FIG. 8.

Stress is term used to denote a broad spectrum of interoceptive, nociceptive and exteroceptive factors which increase stress load and trigger a neurological stress response, also known as the inflammatory stress response and the inflammatory reflex. Modern biofeedback modalities such as galvanic skin response (GSR), peripheral skin temperature, and electromyography can be used to detect biofeedback markers of interoceptive, nociceptive and exteroceptive events that indicate or elicit stress. These markers can be monitored and used to indicate the need for neurostimulation parameterized for stress therapy or may be incorporated into one or more algorithms whereby they automatically trigger such neurostimulation programs. Cardiologic activity can also be monitored as biofeedback using photoplethysmography. Recent research suggests that neurostimulation as a stress therapy targeting the parasympathetic nervous system may be optimized by gating its delivery during periods of parasympathetic activity which may be detected by photoplethysmography. Other devices and methods attempt this by gating stimulation according to respiratory activity. A more economical approach we propose is monitoring heart rate and heart rhythms to detect the activity directly and more accurately indicative of said parasympathetic nervous system activation, such as the heart beat rhythm known as respiratory sinus arrhythmia (RSA), which is known an in index of both relaxation and sympathetic nervous system activation. Unlike respiration itself, respiratory sinus arrhythmia represents an optimal level of sympathetic nervous system activation. Respiratory sinus arrhythmia occurs when a certain style of breathing is executed, causing the heart to accelerate during inspiration and decelerate during expiration. During normal breathing, the heart maintaining a relatively constant rhythm known as normal sinus arrhythmia, which is dominated by sympathetic control of cardiologic activity. To take advantage of the optimizing effects of delivering neurostimulation during periods of elevated parasympathetic nervous system activation, stimulation may be open-gated to coincide with the deceleration of heart beats associated with respiratory sinus arrhythmia. Similarly, stimulation can be close-gated or re-parameterized during periods when pathological arrhythmias are detected. When respiratory sinus arrhythmia is used for open-gating neurostimulation, the breathing method known to produce it can be taught to the end-user or can be prompted by electronic devices, such as mobile applications and other means of behavioral prompting.

Heart rate variability (HRV) is another biofeedback marker that can be used to monitor the activity of the parasympathetic nervous system, as a marker indicating the need neurostimulation and stimulation parameterization. The lower frequency domains of HRV are said to provide indication of the healthy versus pathologic parasympathetic activity and autonomic balance, and can be monitored accordingly.

Self-administered neurostimulation is an emerging modality made possible by the development of small, portable neurostimulation systems like the present invention. Many people including healthcare practitioners will erroneously assume that showing a patient how to use a neurostimulation device, or merely making usage instructions available on the internet, will be all that is required for self-administered neurostimulation training and even some individual users may scoff at the notion of adhering to a self-neurostimulation methodology especially if said methodology requires formal training. However, many such end-users will be employing neurostimulation for disorders and conditions involving significant habitual behavior which will foreseeably impede or block the adoption of neurostimulation and its replacement of the habitual problem behavior associated with the target disorder or condition. Substance addiction is an obvious example. The substance addict uses drugs or alcohol because the acquisition and use of the drug have become habituated into the user's behavioral routine which is negatively reinforced through pathological learning known to disrupt and reprogram certain neurocircuits. The user's habitual routines are typically highly resistant to change even when alternatives to the addictive substance are readily available. It is necessary therefore to determine the conditions required to break the chain of habitual behavior and replace the use of and reliance on substances with the neurostimulation therapy. Post-addiction treatment, over 85 percent of addicts report that acute stress triggers relapse. Addicts can be trained, therefore to recognize the interoception-maker experienced as acute stress as an indicator of the need to use neurostimulation. Contemporary addiction treatment programs train addicts to identify exteroception-markers or external factors which act as stressors to trigger habitual substance abuse. The experience of stress is a primary driver of addiction and experts agree that addiction is stress-driven disease. Hence, the relief of acute stress should be a primary objective of therapy. The stimulation of cranial nerves in the auricular nerve field has been shown to provide relief for acute stress as traditional auricular acupuncture and as modern electro-acupuncture of the auricle. Those recovering from addiction can be trained to self-administer neurostimulation in response to interoception and exteroception makers known as “triggers,” to relieve acute stress instead of using substances.

An additional factor in self-administered neurostimulation is the fact that many conditions and disorders for which neurostimulation may be used present with a pattern of co-morbid symptoms. Mood disorders, for example, often presents with insomnia, eating disturbances, ADHD/ADD, anxiety and the like. PTSD often presents with a similar pattern of co-morbid symptoms and disorders that originate as components of the inflammatory response to stress. A typical middle-aged patient or user of self-administered neurostimulation may therefore need to use neurostimulation in the morning for early morning depression and brain fog. In the afternoon, a different neurostimulation program may be needed for anxiety or ADD. In the early evening another stimulation program may be used for relaxation after work. And in the later evening hours another neurostimulation program may be used to prevent primary insomnia. To provide patients or users with neurostimulation devices without the benefit of training, transfer of training and without training in stimulation parameterization specifically designed to eliminate and minimize barriers to use is to set said patients or users up for failure.

To maximize the benefits of self-stimulation and minimize barriers to adoption and use, the self-administered neurostimulation method therefore incorporates these key principles. The first principle is the elimination or minimization of all possible barriers to performing self-administered stimulation. Self-administered neurostimulation must therefore be extremely quick and easy to set up and use. A second principle is that user-focused training and repeated practice in self-administered neurostimulation is essential to successful adoption of self-administered neurostimulation as a therapeutic modality. To the extent practical, training must include education about the disorder, disease or condition for which self-administered neurostimulation is to be applied; the autonomic nervous system and the rationale for using neurostimulation as a therapeutic intervention or co-intervention. A third principle is that self-administered neurostimulation should be relevantly embedded in the patient's or user's schedule of daily activities and paired and queued to such activities, or alternatively to a time schedule for use, e.g., morning, after work and before bedtime. Each user's symptom pattern should be analyzed and mapped according to interoception and exteroception markers, and, when biofeedback devices are used, biofeedback markers associated with target symptoms (e.g., stress, depression, anxiety, ADD). Symptom mapping provides the framework for integrating and embedding self-administered neurostimulation in the real world daily experiences of the user.

A fourth principle is that the self-neurostimulation should be reinforced by third-party healthcare professionals or caregivers. Caregivers should participate in self-administered neurostimulation training. Reinforcement involves knowing the aforesaid symptom map of the patient and using identified interoception, exteroception and biofeedback markers to cue-up reminders to initiate self-administered neurostimulation and neurostimulation program selection. Reinforcement of self-administered neurostimulation may be accomplished electronically, via SMS text messages, via telephone, or by a reinforcement program residing on a computerized device such as a smartphone or tablet, or as a program within the neurostimulation system itself.

A fifth principle of self-administered neurostimulation is to maximize the availability, usability and accessibility of the user interface for operating the neurostimulation device. The user interface must make the selection and operation of the stimulation program as automatic and simplistic as possible. Stimulation program selection should be via graphic selection means. For example, a stimulation program for insomnia is operated by a selector having an icon for sleep, e.g., a crescent moon. When multiple programs of self-neurostimulation are available on a device, the user should only decide which program to use and the stimulation intensity level. The parameterization of stimulation programs should be left to professionals or to healthcare providers. In most cases, a single selector can function to start the stimulation program, pause it, resume it, and stop it by way of multiple button-presses or similar selection activity.

### BRIEF DESCRIPTIONS OF THE DRAWINGS

FIG. 1A Stimulation apparatus, basic unit with stimulation cable.

FIG. 1B Stimulation apparatus, worn on human ear model 140, indicating dorsal component 104 and ventral module 104 inserted to the cymba concha 145 in order to stimulate the vagus nerve through the cavum concha nerve zone 163.

FIG. 1C Stimulation apparatus, self-contained wireless unit with dorsal side energy emitters.

FIG. 2A Human ear anatomical features as indicated 141 through 154.

FIG. 2B Human ear auricular nerve field indicating target nerve field zones.

FIG. 2C Human ear dorsolateral view indicating nerve targets including as indicated 165 through 169.

FIG. 3 Stimulator block diagram.

FIG. 4A Spherical magnet.

FIG. 4B Cylindrical magnet with diametric polarity.

FIG. 4C Cylindrical magnet with axial polarity.

FIG. 4D Block magnet with axial polarity.

FIG. 4E Block magnet with planar polarity.

FIG. 5A Block diagram with biofeedback to user interface.

FIG. 5B Block diagram with biofeedback to stimulator unit.

FIG. 5C Block diagram with emitter/biofeedback sensors multiplexed to stimulator unit.

FIG. 6 Biofeedback controlled neurostimulation block diagram.

FIG. 7 Temporal periods of stimulation and therapeutic intervention.

FIG. 8 Yerkey-Dodson Law of optimal arousal applied neurostimulation.

### REFERENCE NUMERALS IN DRAWINGS

100 Ear worn stimulation apparatus
101 Dorsal body
102 Ventral module (pod)
103 Ventral connection cable
104 Dorsal body crotch energy emitter coupler
105 Ventral module energy emitter coupler
106 External stimulator unit to dorsal body cable
107 Ventral stimulation trigeminal nerve zone energy coupler
108 Ventral stimulation lesser occipital nerve zone energy coupler
110 Computer platform
111 Communications interface
112 Stimulator unit
113 Stimulator controller
114 Stimulator energy monitor
115 Stimulator signal output
116 Protocol data table
117 Communication I/O
118 Processor program memory
119 Analog input
120 Waveform generator
121 Voltage monitor signal conditioning
122 Current monitor signal conditioning
123 Voltage control amplifier
124 Current control amplifier
125 Biofeedback sensor signal conditioning
126 Multiplexer
127 Ear worn stimulator emitter/biofeedback sensor couplers
128 Emitter to user skin coupling and tissue resistance
140 Human ear
141 Dorsal apex (farside)
142 Dorsal crotch (farside)
143 Cura of antihelix
144 Scapha
145 Cymba concha
146 Antihelix Cavum
147 Helix
148 Concha
149 Antitragus
150 Lobule
151 Cavum
152 Tragus
153 External auditory meatus
154 Crus of helix
155 Triangular fossa
160 Auricular nerve field
161 Trigeminal (v.3) nerve zone
162 Lesser occipital nerve zone
163 Cavum concha
164 Great auricular nerve zone
165 Dorsolateral trigeminal nerve target V1
166 Dorsolateral auricular branch vagus nerve target V2
167 Dorsolateral lesser occipital nerve target V3
168 Dorsolateral auricular branch vagus nerve target V4
169 Dorsolateral great auricular nerve target V5
201 Magnet
202 Spherical magnet
203 Cylindrical magnet indicating diametric polarity
204 Cylindrical magnet indicating axial polarity
205 Block magnet indicating axial polarity
206 Block magnet indicating planar polarity
230 Biofeedback sensor array
231 Biofeedback photoplethysmography sensor
232 Biofeedback body/limb position sensor
233 Biofeedback body motion sensor
234 Biofeedback response exception circuit
235 Biofeedback body acceleration
236 Biofeedback body position and change rate algorithm
237 Biofeedback limb motion and change rate algorithm
238 Biofeedback control algorithm supervisor
239 Biofeedback control algorithm knowledge base
240 Biofeedback control algorithm determinants
250 Pre-intervention
251 Neurostimulation enhanced therapeutic pre-intervention phase 1 relaxation
252 Neurostimulation enhanced therapeutic pre-intervention phase 2 warm-up
253 Therapeutic intervention
254 Neurostimulation enhanced therapeutic intervention phase 3 exercise
255 Neurostimulation enhanced therapeutic intervention phase 4 rest
256 Neurostimulation enhanced therapeutic intervention phase 5 training
257 Post-intervention
258 Neurostimulation enhanced therapeutic post-intervention phase 6 recover
259 Neurostimulation enhanced therapeutic post-intervention phase 7 normalize

### Apparatus Embodiments

Disclosed is an invention comprising an auricular neurostimulation system having modular components for selectively targeting one or more nerves and for selecting and applying the type of energy to be used for stimulation. The invention features removable coupling means for accomplishing secure and consistent positional contact of energy emitters and biofeedback sensors applied to skin surfaces overlaying the auricular nerve field of the human ear. Benefits of the invention include comfortable wearability, rapid attachment and removal, easy electrode positioning and superior attachment security.

The present invention couples energy emitter or electrodes to the skin of the human ear without adhesives or spring-actuated clamps, while employing compressive force sufficient to maintain the positions of the energy emitters and sensors relative to target nerves and sensor targets in all bodily orientations and during normal body movement including light exercise. In the present invention, said coupling is achieved by means of the coupling attraction force between a pair of magnets or a magnet and associated coupled ferromagnetic material.

In addition to providing consistent compressive contact and nerve intersecting positional alignment, the magnetic coupling between the dorsal and ventral sides of the ear provide positional stability of the ear-worn stimulation coupling apparatus.

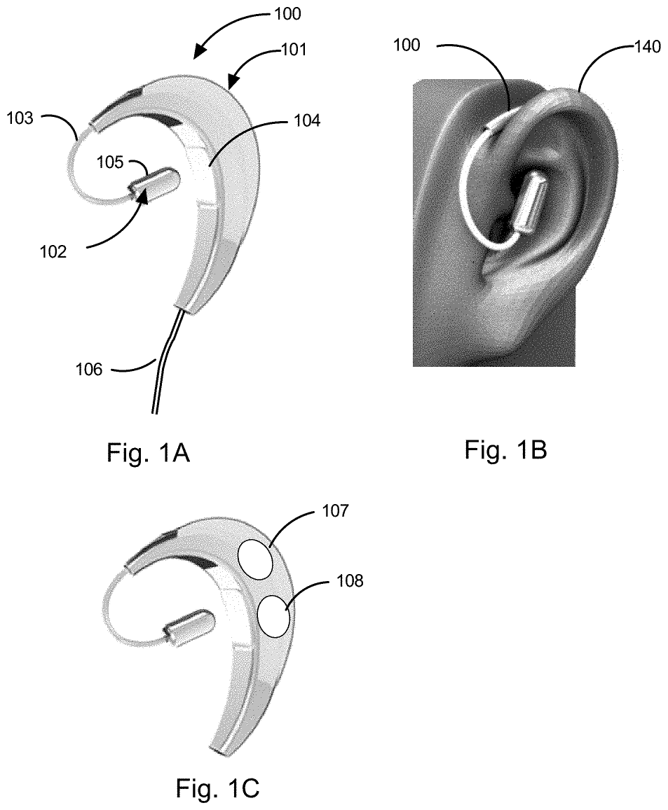

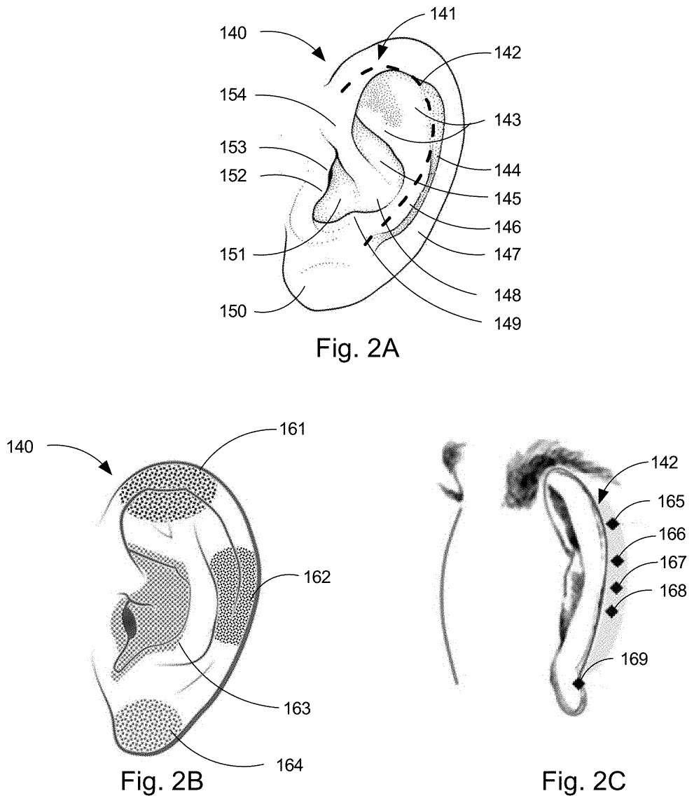

In one exemplar embodiment, wherein stimulation of the auricular branch of the vagus nerve (ABVN) is exemplified, a representative stimulation apparatus 100 having a dorsal body 101 is worn over the dorsal-dorsolateral crotch of the ear 142 and a ventral module component 102 inserted as convenient in the cymba conchae concha trench 145 as shown in FIG. 1A. Said stimulation apparatus as shown in FIG. 1A and FIG. 1C consists of a dorsal body 101 and associated ventral module 102 each containing at least one magnet or an associated ferromagnetic material as convenient to affect magnetic attraction by opposing magnetic poles. Said dorsal body and ventral module components each have associated energy emitter contacts 104 and 105. A ventral module connection cable 103 provides mechanical attachment and electrical connectivity between said dorsal body component and said ventral module coupler. Said ventral module coupler and dorsal body components can be conveniently manufactured utilizing 3D or plastic injection molded materials and conventional processes.

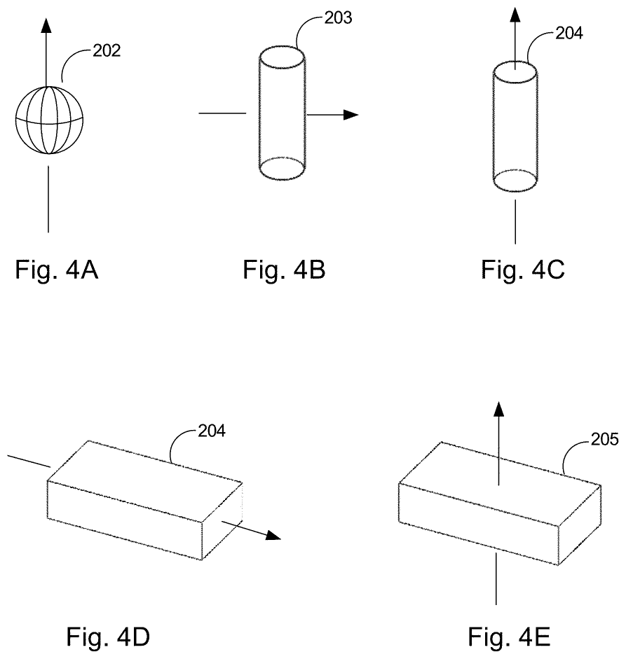

Magnet components configured as shown in FIG. 4A through FIG. 4E are conventionally and readily available in a variety of sizes and magnetic forces. Magnet shapes include: spherical 201; cylindrical with diametric polarity 202; cylindrical with axial polarity 203; block with axial polarity 204; and block with planar polarity 205. Experimental prototypes utilizing magnets with paired contact attractive forces in the order of 2 to 5 pounds provide the necessary compression to effect the desired attributes of consistent and reliable energy stimulation contact, comfort and secure positioning. Additionally, said magnetic components can readily be custom manufactured in specified shapes, polarity and magnetic forces.

It is well known that characteristic coupling force between magnetic coupling is reduced according to the reciprocal of the distance squared (F=1/(D\hat{ }2). Various magnet configurations may be utilized within the loop and ventral components in order to optimize compression forces upon selected nerve field ear tissue surfaces. Such configurations in various embodiments may include: different shapes, sizes and force characteristics; mechanically altering the distance between magnet components; mechanically altering polar orientation of magnets. Each of these configurations are considered with respect to the design goal to optimize the positions energy coupling emitters and biofeedback sensors as well as the comfort of the user.

Various design features may be incorporated as convenient in the structural design and manufacture of ventral and loop components to mechanically alter the position, orientation and distance factors. Using experimental prototype devices for the represented vagus nerve stimulation, the inventors have determined that optimized positional contact, ease of use and wearing comfort is achieved by designs that allow the magnet within the dorsal loop component to self-align magnetic force vectors in polar orientation and also to translate with respect to placement of said ventral module coupler. This has been tested and demonstrated in various prototypes incorporating spherical, block and cylindrical type magnets.

In a basic embodiment, the ear-worn stimulation coupling apparatus 100 includes a dorsal body component 101 and ventral module component 104 mechanically and electrically connected by means of connecting cable 110. Said dorsal body provides the mechanical structure to include at least one dorsal magnet 102 and at least one energy emitter. Said ventral module provides the mechanical structure to include at least one magnet and at least one energy emitter coupler 105.

Additionally, said dorsal body magnet and ventral module magnet may conveniently incorporate any one of or combination of the spherical, cylindrical and block types as indicated in FIG. 4A through FIG. 4E and be manufactured from various strength magnets or ferro-magnetic material as suitable.

Additionally, said dorsal body may incorporate at least one coupler(s) 104 and said ventral module components 102 contain at least one said energy emitter coupling as indicated in FIG. 1A and FIG. 1C. Said energy emitter couplings providing electrical stimulation are manufactured from an electrically conductive material. Said electrically conductive materials include conductive ink, graphene, epoxy, plastic or metal surfaces and the like as convenient. Further, said electrically conductive material used for electrical contact with the skin may be plated with a noble, hypo-allergenic material such as gold, silver, palladium and the like. In the case of electric energy stimulation, said dorsal and ventral energy emitter couplers are nominally aligned such that electrical current path intersects the target nerve and/or nerve field to be stimulated.

Additionally, at least one said electrically conductive coupling contact surface may be a magnet or ferromagnetic material.

Additionally, at least one said magnet configured as an electrically conductive contact may be positionally adjustable in order to optimize proximity to a target nerve.

Additionally, said at least one dorsal body coupler(s) 104 and/or said at least one ventral module coupler(s) 107 may comprise photo-optical emitters. In such case the target nerve and/or nerve field to be stimulated lies beneath the contact of said coupler. In contrast to electrical energy stimulation that requires a conduction path between two electrical contacts through ear tissue, photo-optical emitted energy may be deposited in tissue from single point emitter contact, on the dorsal or the ventral side of the ear, as convenient, or on both sides of the ear if further research indicates advantages and/or benefits of multi-point or nerve intersecting photo-stimulation.

Additionally, said at least one dorsal body coupler(s) 104 and/or said at least one ventral module coupler(s) 102 may incorporate electromechanical-vibrational or piezoelectric-acoustic emitters. In such case the target nerve and/or nerve field to be stimulated lies beneath the contact of said coupler. In contrast to electrical energy stimulation that requires a conduction path between two electrical contacts, (ear tissue), only a single point energy emitter is required as convenient on the dorsal or the ventral side of the ear, or on both sides of the ear if further research indicates advantages and/or benefits of multi-point or nerve intersecting vibrational stimulation.

Additionally, a mix of electrical and/or photo-optic and/or vibrational energy emitters and couplers may be incorporated in combination.

Additionally, at least two electrical energy stimulation coupler contact poles may be incorporated on either or both of said dorsal body and/or on said ventral module to enable skin surface electrical conduction circuit path.

Additionally, a said ventral energy coupler of photo-optic type may integrate a photo-emitter and therefore connect to said dorsal body electromechanically by means of an electrical cable to said stimulation generator. Alternatively, said ventral photo-optic emitting energy coupler may connect photo-optically to said stimulation generator by means of a fiber optic cable with said photo-emitter located in said dorsal body.

Additionally, biofeedback sensors may be included as optical sensors configured for photoplethysmography. Said biofeedback sensors may be incorporated in either said dorsal body worn behind the ear, in a said ventral module worn of the ventral surface of the ear, or in both utilizing a proximity type, single sided photo-emitter pair, or a through-beam type. Said biofeedback may utilize discrete sensor components, or be designed as part of an energy stimulation coupler, for example whereby the photo-optic stimulation emitter also functions as a photoplethysmography emitter.

Additionally, said photoplethysmography emitter and/or detector may utilize fiber optic signal transfer between the target skin surface and the photo-electronic emitter and detector device.

Additionally, electrical sensors may be included as to monitor one or more types of electrical activity such as electrical conduction through the tissue between the dorsal and ventral sides of the ear; the electrical conduction across either or both sides of the skin surfaces of the dorsal and/or ventral; and the electric field strength as occurring proximal to the auricular nerve field areas.

The energy stimulation electronics package embodiments include a configuration wherein the electronics package is directly wired to the dorsal body by means of a cable 106 as shown in FIG. 1A or as an integrated, embedded version as shown in FIG. 1C. The directly wired version includes a multi-conductor cable from connected from an electrical stimulation unit to the dorsal body. In such embodiment, electrical connections are hardwired to selected energy stimulation emitters and/or sensors located in the dorsal body and/or ventral module couplers, as convenient. As such, the stimulation unit may be used as a standalone device to select operational protocols. The integrated, wireless ear-worn version necessarily includes a battery power source, power supply, microcontroller, wireless communication, and low level real-time stimulation generation and waveform synthesis electronics within the dorsal body as shown in FIG. 1C. As such, high level control features are provided by means of a personal wireless enabled computing platform 110 with high level user interface as conveniently provided by a conventional smart phone, tablet or the like.

Additionally, stimulation protocols settings including stimulation frequency, voltage, current, waveforms, session duration, session scheduling and the like are set by the user by means of a stimulation control unit whereby the generated stimulation signals are directly connected to the ear-worn dorsal body and/or ventral module couplers. In the case of a wireless connection between a personal computing platform and a wireless embedded unit, said stimulation protocol settings are set using said computing platform and transmitted as a data set to be executed under real-time control of an embedded controller.

Additionally, said control electronics of both the wired and wireless control embodiments incorporate current and voltage feedback to monitor and regulate the stimulation energy applied to the user according to set points as determined by an algorithm, directly input to the stimulation controller by the user, or received by remote download.

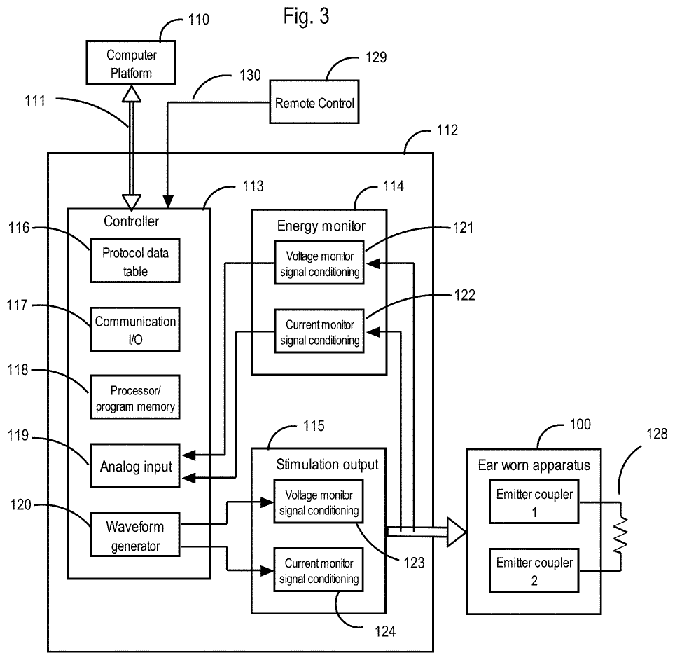

FIG. 3 is a block diagram illustrating an embodiment of a control system including a computer platform 110 used as a preferred means for user operational interface, to process and store control protocols for download to the stimulator unit 112 and to upload, process and store data from biofeedback sensors and to communicate with the internet, and the like. Said computer platform communicates with said stimulator unit by means of a local wireless communication interface 111 such as Bluetooth or the like. Said stimulator unit contains a controller based on processor such as a microcontroller with program memory 118, with memory allocation for protocol data table 116, communication I/O 117, analog input and signal conditioning 119 and waveform generator 120. Said stimulator unit also incorporates electronic circuitry such as voltage and current regulation amplifiers to provide stimulation signal output 115 to provide stimulation signals to the ear worn apparatus 100. Said stimulation unit also features an energy monitor 114 with signal conditioning electronics to input and condition output voltage 121 and current 122 thereby providing closed loop feedback to the controller electronics analog input in order to adjust the signals from the waveform generator. Said signal output feedback monitoring is advantageous in order to compensate for electrical resistance variations 128 realized by the ear worn apparatus 100 emitter couplers making contact with the user's skin.

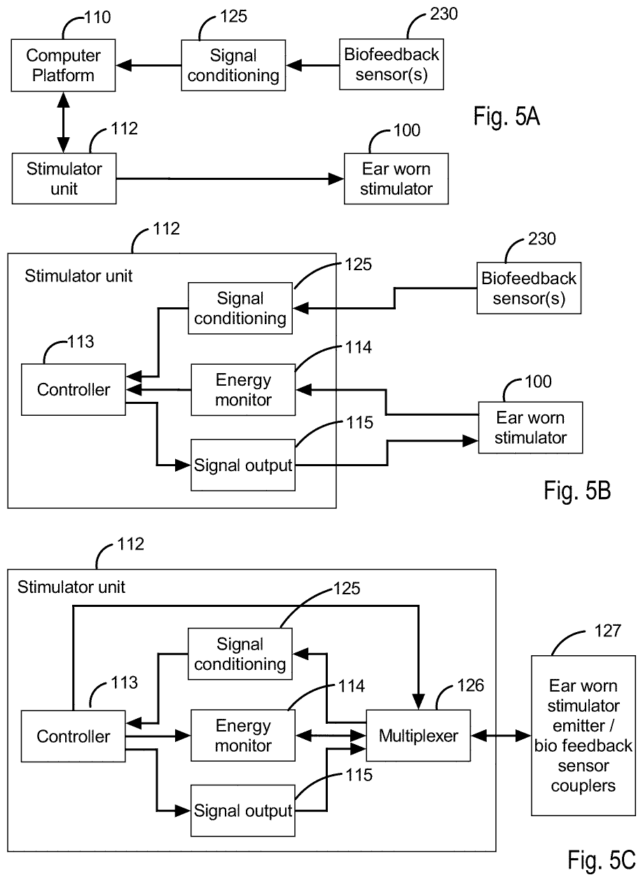

FIG. 5A illustrates an embodiment incorporating a stimulator unit 112 connected to an ear worn stimulator apparatus 100. Said stimulator unit 112 communicates with a computer platform 110 receiving input from a standalone, external signal conditioning electronics package 125 inputting biofeedback sensor(s) 230.

FIG. 5B illustrates a stimulator unit 112 illustrating the integration of a controller 113, biofeedback signal conditioning electronics 125, energy monitoring electronics 114 and signal output 115. Biofeedback sensor(s) 230 are distinct and may be incorporated as part of the ear worn apparatus, or be externally worn by the user on various parts of the body.

FIG. 5C illustrates a an embodiment of a stimulator unit 112 integrating a controller 113, biofeedback signal conditioning electronics 125, energy monitoring electronics 114 and stimulation signal output electronics 115. Multiplexing electronics 126 time phases the switching of stimulation signal, energy monitoring signals and biofeedback signals under the control of said controller. Said multiplexer thereby enables the dual use of ear worn stimulator emitters with biofeedback sensor couplers 127. For example, such said emitter/sensor couplers may be photonic emitters also used for the purpose of photoplethysmography. Additionally, electrical emitters may serve as electrical sensor contacts to detect determined signals based on skin impedance, for example.

Additionally, said dorsal body incorporates at least one connector port for cable connection to at least one interchangeable ventral coupling module as convenient. Said connector port and associated cable serve to interface at least one and/or a combination of electrical, photonic or acoustic stimulation energy and/or biofeedback type signals between said dorsal body and said ventral module wherein electrical energy utilizes and electrical conductor, photonic energy utilizes optical fiber and acoustic energy utilizes an acoustic waveguide.

Additionally, said dorsal body and at least one ventral module may be conveniently manufactured utilizing conformable plastic material such as silicone rubber, ABS and the like, with appropriate durometer selected for form, fit and function in order to optimize wearing comfort and proper positioning of said coupling emitters. Said dorsal body and said ventral module components can also be overmolded or applied with secondary materials as well as conformable materials overmolded on stiffening structures as convenient. Said stiffening structures may also utilize materials including plastics, metals and/or compositions designed to stiffen as desired using temperature or photonic actuation.

### Method Embodiments

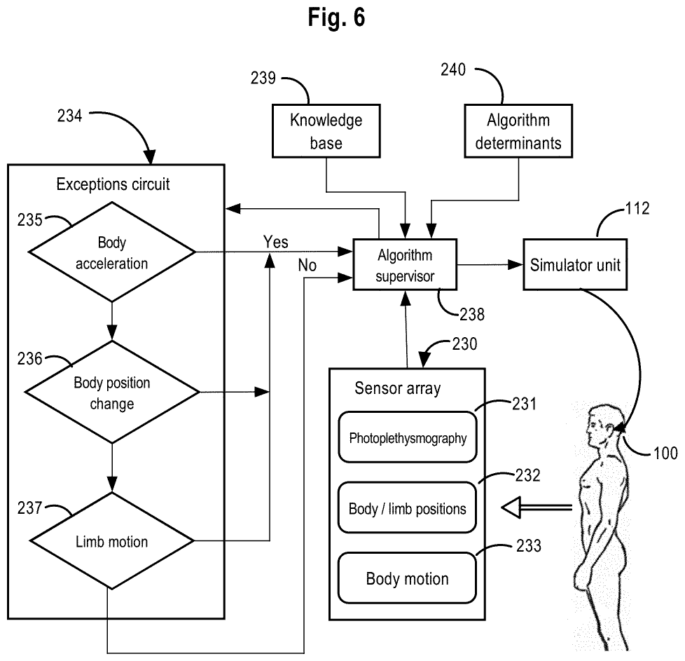

In a further embodiment of the present invention, methods are employed to utilize the apparatus and data derived from user thereof In one method embodiment as illustrated in FIG. 6, the user with the ear worn stimulation apparatus 100 is monitored by a sensor array 230 providing anatomical biofeedback including photoplethysmography 231, body and/or limb positions 232 and body motion 233. Said anatomical biofeedback has been found by research to validate or invalidate the usefulness of other stimulation efficacy measures such as heart rate variability. As shown in FIG. 6, said sensory information is received and processed by an algorithm supervisory program 238. Said processed information is then received by an exceptions circuit program to examine said information to determine the user's body acceleration 235, changes in body position change 236 and limb motion 237. The results of said exception circuit analysis is then handled by the algorithm supervisor 238 utilizing information data from a knowledge database 239 and algorithm determinants 240 in order to provide any necessary adjustments to the stimulator unit 112 and thereby affect the stimulation process for the user.

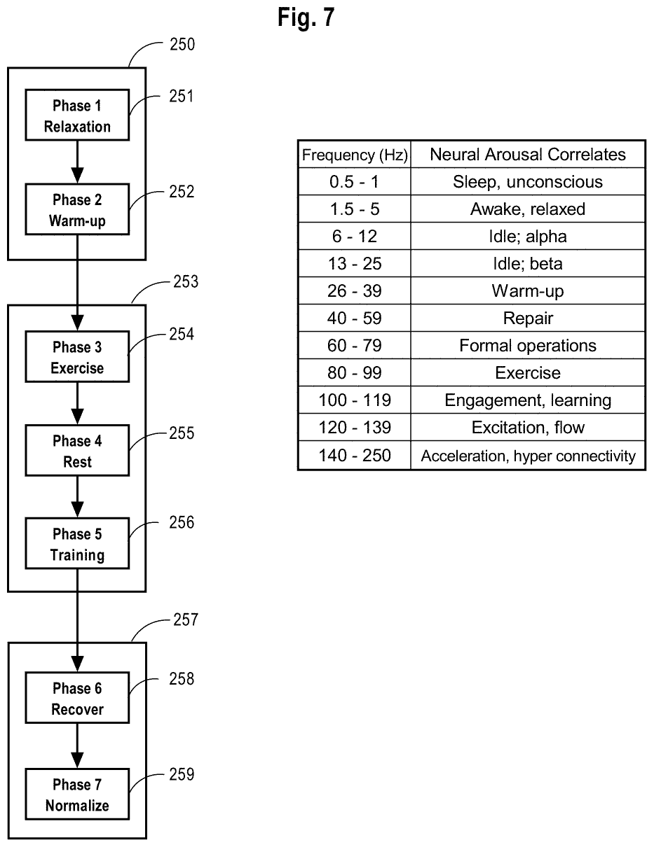

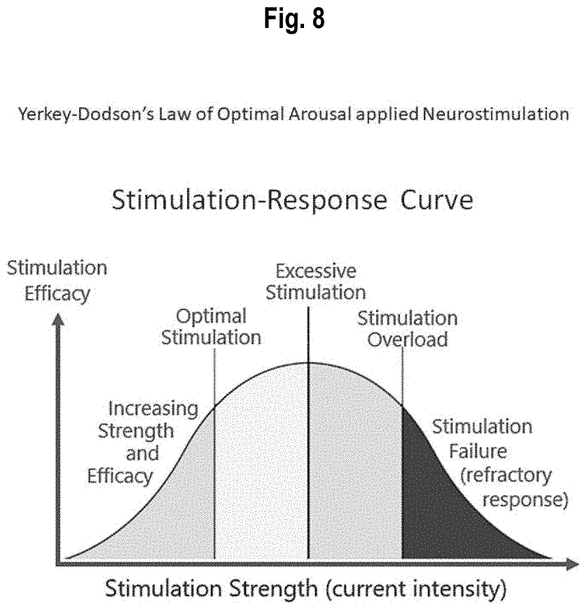

A further methodology embodiment of the present invention include neurostimulation enhanced therapy procedures as illustrated in FIG. 7 which describes a sequence of grouped process phases administered over a period of time and includes a table of neural arousal correlates. A properly designed personal stimulation program would enable a therapist and user to construct, for example, a daily schedule of stimulation sessions to enhance the user's wellbeing. These correlates illustrate, as based on current research, recommended frequency ranges to be applied according to each of the group phases. A pre-intervention group 250 involves phase 1 relaxation 251 and phase 2 warm up 252; a therapeutic intervention group 253 involves phase 3 relaxation 254, phase 4 rest 255 and phase 5 training 256; and a post intervention group 257 involving phase 6 recover 258 and phase 7 normalize 259.

The first step in practical application requires a therapist to plan and map the key phases of the proposed therapy from each of the said groups. The second step is to compose or select from a pre-composed scale of therapeutically relevant neural arousal correlates as shown in the table in FIG. 7 an indexed continuum of neurostimulation frequencies. In the third step, each phase of the therapeutic intervention neurostimulation parameters are selected to provide the most beneficial neural arousal level per intervention phase. Once composed, said stimulation intervention programs could be then be selected by the user by means of single, easy to use icon button on a personal computing platform, smart phone, or the like.

While various embodiments of the present invention have been described above, it should be understood that they have been presented by of way of example only, and not of limitation. Likewise, the various diagrams may depict and example configurations for the invention, which is done to aid in understanding features and functionality that can be included in the invention. The invention is not restricted to the illustrated example configurations, but can be implemented using a variety of alternative configurations. Additionally, although the invention is described in terms of various exemplary embodiments and implementations, it should be understood that the various features and functionality described in one or more of the individual embodiments are not limited in their applicability to the particular embodiment with which the are described, but instead can be applied, alone or in some combination, to one or more of the other embodiments of the invention, whether or not such embodiments are described and whether or not such features are presented as being part of a described embodiment. Thus, the breadth and scope for the present invention should not be limited by any of the above described embodiments.

Terms and phrases used in this document, and variations thereof, unless otherwise expressly stated, should be construed as open ended as opposed to limiting. As examples of the foregoing: the term “including” should be read as to mean “including without limitation” or the like; the term “example” is used to provide exemplary instances of the item in discussion, not an exhaustive or limiting list thereof; and the adjectives such as “conventional”, “traditional”, “normal”, “standard”, “known” and terms of similar meaning should not be construed as limiting the item described to a given time period or to an item available as of a given time, but instead should be read to encompass conventional, traditional, normal or standard technologies that may be available or known now or at any time in the future. Likewise, a group of items linked with the conjunction “and”, should not be read as requiring that each and every one of those items be present in the groupings, but rather should be read as “and/or” unless expressly stated otherwise. Furthermore, although items, elements or components of the invention may be described or claimed in the singular, the plural is contemplated to be within the scope thereof unless limitations to the singular is explicitly stated. The presence of broadening words and phrases such as “one or more”, “at least”, “but not limited to” or other like phrases in some instances shall not be read to mean that the narrower case is intended or required in instances where such broadening phrases may be absent.

We claim:

1. An improved, modular energy stimulation system comprising apparatuses including energy stimulation means for generating and conditioning stimulation energy, controller means for controlling said energy stimulation means, magnetic coupling means for coupling energy emitter means to at least one ear of a human being and applying energy stimulation to at least one cranial auricular nerve accessible in the auricular nerve field of a said human being, wherein:
said energy stimulation means includes a stimulator for generating and conditioning energy for stimulation, a power source, power control and recharging electronics, and at least one channel of stimulation energy output;
said magnetic coupling means providing magnetic coupling force sufficient to securely and conductively couple at least one said energy emitter means to the said at least one auricle of a said human being;
said magnetic coupling means is configured for coupling a said at least one energy emitter means to the auricle of a human being;
said at least one energy emitter means is selected from a group that includes emitters of electrical energy, optical emitters of electromagnetic energy, and emitters of vibrational energy;
said controller means is configured to control energy stimulation parameters selected from a group that includes stimulation frequency, waveform, pulse rate, pulse width, amplitude, stimulation duration, stimulation periodicity, and the like;
said stimulation periodicity is comprised of at least one selected time interval of stimulation delivery over a 24 hour period;
said at least one cranial nerve is selected from a group of nerves accessible in the auricular nerve field that includes the auricular branch of the vagus nerve, the auriculotemporal nerve, the lessor occipital nerve, and the great auricular nerve also known as the greater auricular nerve.

2. The system of claim 1, wherein:
said at least one magnetic coupling means is comprised having at least one magnet and at least one magnetic composition;
said magnetic coupling means is comprised having interchangeability means to enable the removal and replacement of magnetic compositions of different magnetic strengths according to the coupling force preferred a human user;
at least one said magnetic coupling means and at least one said energy emitter means are configured as an at least one ear-worn coupling apparatus;
said at least one ear-worn coupling apparatus is comprised having a dorsal body worn behind the auricle against the dorsal-dorsolateral crotch of the auricle of a human being;
said at least one ear-worn coupling apparatus having a weight between 2.5 and 120 grams.

3. The system of claim 2, wherein:
said at least one ear-worn coupling apparatus is be further comprised having at least one ventral coupling module including at least one energy emitter means;
said at least one ventral coupling module is worn having contact with the front, ventral-ventrolateral surface of the auricle;
said at least one ventral coupling module is further comprised having at least one magnetic composition for coupling the ear between said dorsal body and said at least one ventral coupling module;
said at least one ventral coupling module is electronically connected to said dorsal body by at least one adjustable conductor means;
said at least one adjustable conductor means having adjustability features belonging to a group of adjustability features including positionability, rotatability, flexibility, bendability and the like;
said controller means is configured for the selective control of at least two stimulation parameters belonging to a group of stimulation parameters that includes power amplitude, fluence, waveforms, wavelengths, pulse widths, phase characteristics, stimulation channels, stimulation frequencies, stimulation session periods, signal duty cycle, periodicity of stimulation, total energy delivered and the like.

4. The system of claim 3, wherein:
said at least one ventral coupling module is configured for contact with the ventral-ventrolateral surfaces of the auricle;
said dorsal body is comprised having at least one magnetic composition;
said dorsal body and said at least one ventral coupling module are configured for magnetically coupling said at least one energy emitter to the auricle of a human being;
said dorsal body is further comprised having at least one connector port for connecting said at least one adjustable conductor means;
said at least one connector port on said dorsal body connects said dorsal body to said at least one ventral coupling module via said adjustable conductor means;
said adjustable conductor means is comprised of conductive material selected from a group that includes metal wire, conductive plastic material, conductive elastomers, and the like.

5. the system of claim 4, wherein:
said at least one ventral coupling module and said dorsal body, each having at least one electrical energy emitter, comprise an electrical energy emitter circuit;
said electrical energy emitter circuit is configured to be coupled to the said ventral-ventrolateral and said dorsal-dorsolateral surfaces of a said person's said auricle;
said electrical energy emitter circuit is configured to spatially and anatomically intersect said at least one auricular nerve target;
said nerve intersection comprises the positioning of a said at least one energy emitter circuit's energy emitters on opposing ventral-ventrolateral and dorsal-dorsolateral surfaces of the auricle wherein a said at least one target nerve lies in-between.

6. The system of claim 5 wherein:
said at least one ventral coupling module includes energy emitters selected from a group that includes emitters of electrical energy, optical emitters of electromagnetic energy, and emitters of vibrational energy;
said dorsal body includes energy emitters selected from a group that includes emitters of electrical energy, optical emitters of electromagnetic energy, and emitters of vibrational energy;
said emitters of electrical energy are configured to emit electrical stimulation current having at least one electrical frequency selected from a range of electrical frequencies between 0.5 Hertz to 250 Hertz;
said optical emitters of electromagnetic energy are selected from a group of optical emitters that includes LEDS, OLEDS, VCSELS, optical graphene emitters and the like;
said optical emitters of electromagnetic energy emit electromagnetic energy having wavelengths selected from a range of wavelengths between 400 and 1600 nanometers;
said optical emitters of electromagnetic energy are configured to emit electromagnetic energy having a power density selected from the range between 0.5 and 25 joules per square centimeter;
said emitters of vibrational energy are configured to emit vibrational energy at frequencies of 1 Hertz to 10,000 Hertz.

7. The system of claim 6, further comprising:
at least one computerized device selected from a group that include a conventional desktop computer, a notebook computer, a laptop computer, a smartphone, a tablet, a handheld computer and a wearable, user-attached computerized device;
a graphical user interface configure to provide selective control of at least two stimulation parameters belonging to a group of stimulation parameters that includes power amplitude, fluence, waveforms, wavelengths, pulse widths, phase characteristics, stimulation channels, stimulation frequencies, stimulation session periods, signal duty cycle, and time intervals of stimulation delivery, stimulation periodicity, total energy delivered and the like;
said at least one computer device having hardware configured for electronic communication between said at least one computer device and said stimulator, said hardware configured for said electronic communication selected from a group that includes hardware for wired communication and hardware for wireless communication;
said communication hardware and said software installed on said at least one computer device to enable internet connectivity and the communicative exchange of data with at least one remote server.

8. The system according to claim 7, further comprising:
at least one biofeedback sensor means configured for sensing biological signals and biological functioning of a said human being;
at least one bodily activity sensor means configured for sensing the movement and bodily position of a said human being;
said at least one biofeedback sensor means selected from a group that includes sensors configured for photoplethysmography, skin conductance monitoring, electromyographical monitoring, and the like;
said photoplethysmography configured to monitor at least one cardio-respiratory parameter belonging to a group that includes heart rate, respiratory rate, heart rate variability, arrhythmia, normal sinus rhythm, oxygen saturation and blood pressure;
algorithmic control means configured for controlling the delivery of energy stimulation in accordance with algorithm determinants and data obtained from said biofeedback sensor means and said bodily activity sensing means.

9. The system according to claim 8, wherein:
said photoplethysmograph monitoring is configured to detect cardiologic activity of a user, particularly normal sinus rhythm, pathological arrhythmias, and respiratory sinus arrhythmia also known as RSA;
said detection of said respiratory sinus arrhythmia is accomplished by monitoring to detect the start and end of periods of said respiratory sinus arrhythmia;
said photoplethysmograph monitoring is configured to detect the normal sinus rhythm of the heart beats of a user;
said detection of said normal sinus rhythm is accomplished by monitoring to detect the start and end of periods of said normal sinus rhythm;
said photoplethysmographic monitoring of said respiratory sinus arrhythmia is used to gate stimulation according to detected said respiratory sinus arrhythmia;
said photoplethysmographic monitoring of said normal sinus rhythm is used to gate stimulation according to detected said normal sinus rhythm;
said photoplethysmographic monitoring of said cardiologic activity of a user is used to gate stimulation according to detected pathological heart rhythms known as arrhythmias;
at least one algorithm is composed for selectively controlling stimulation parameters according to algorithm determinants and monitored bodily-generated, sensor-received data selected from a group of sensors means that includes said photoplethysmography sensor means, said biofeedback sensor means, and said bodily activity sensor means.

10. The system according to claim 9, wherein:
said photoplethysmography sensor means is further configured to monitor at least one index of autonomic nervous system activity;
said photoplethysmographically monitored at least one index of autonomic nervous system activity includes one or more heart rate variability (HRV) frequency domains selected from a group that includes high frequency, low frequency, very low frequency and ratios thereof, and the like;
said movement and bodily position sensor are configured to monitor body position changes and body positions including standing, sitting, reclining, and lying supine, and the like;
said movement and bodily position sensors are configured for monitoring the acceleration, motion, and position of the body in whole or in part;
said at least one algorithm is composed for selectively controlling one or more stimulation parameters according to algorithm determinants and said at least one monitored index of autonomic nervous system activity, and data from said biofeedback sensor means and movement and bodily position sensors;
said neurostimulation system is comprised as an algorithm-operated and algorithm-controlled closed-loop system;
said closed loop system is further comprised as a self-contained wearable system having a power source and control electronics integrated within a said at least one dorsal body, or composed within a separate second body having electronic communication with said at least one dorsal body and said at least one ventral coupling module;
said one or more stimulation parameters controlled by said at least one algorithm are selected from a group that includes energy frequency, energy intensity, stimulation time duration, energy pulse width, energy waveform, stimulation duty cycle, power amplitude, fluence, waveforms, wavelengths, pulse widths, phase characteristics, stimulation channels, stimulation session periods, signal duty cycle, periodicity of stimulation, total energy delivered and the like.

11. A method of optimizing therapeutic interventions via neurostimulation, the method comprising:
the use of a neurostimulation system configured for transcutaneous delivery of stimulation to at least one cranial nerve target in the auricular nerve field of a human being;
at least one cranial nerve target is selected from a group of cranial nerves that includes the auricular branch of the vagus nerve, the lesser occipital nerve, the trigeminal nerve and the great auricular nerve also known as the greater auricular nerve;
said neurostimulation system is configured to emit energy from at least one energy emitter selected from a group that includes emitters of electrical energy, optical emitters of electromagnetic energy, and emitters of vibrational energy;
at least one selectable energy modality is selected from a group that includes
electrical stimulation current having at least one electrical frequency selected from a range of electrical frequencies between 0.5 hertz to 250 hertz with a pulse-width in the range from 100 to 500 micro-seconds (μS);
optically emitted electromagnetic energy having at least one wavelength selected from a range of wavelengths between 400 and 1600 nanometers emitted at a frequency of between 1 and 5,000 Hertz;
said optically emitted electromagnetic energy having at least one power density or fluence selected from the range of fluence between 0.5 and 30 joules per square centimeter;
vibrational energy having at least one frequency in the range of 1 Hz to 20,000 Hertz with a pulse-width in the range from 100 to 500 micro-seconds (μS);
said neurostimulation system is configured to produce at least one energy train having controller-modulated stimulation parameters selected from a group that includes energy frequency, energy intensity, energy fluence, stimulation time duration, energy pulse width, energy waveform, and total energy delivered, stimulation periodicity, and the like;
neurostimulation controller functions are operable by means selected from a group of controller function operation means that includes a local human operator, a remote human operator, and at least one algorithm;
said neurostimulation controller functions include the adjustment of at least one stimulation parameter selected from a group that includes energy frequency, energy intensity, stimulation time duration, energy pulse width, energy waveform, stimulation duty cycle, stimulation periodicity, total energy delivered and the like;
said neurostimulation is selectively timed, applied and parameterized in relation to a therapeutic intervention, wherein said relation marker belongs to a group that includes
a temporal marker relative to the delivery of said neurostimulation and a said therapeutic intervention;
at least one exteroception marker targeted by a said therapeutic intervention;
at least one interoception marker targeted by a said therapeutic intervention;
at least one biofeedback marker target by a said therapeutic intervention;
a hybrid combination of said markers.

12. The method of claim 11, wherein a said temporal relation marker is based on timing of neurostimulation relative to a said therapeutic intervention;
said neurostimulation is delivered for a period of time occurring in at least one of the temporal periods selected from a group that includes:
a pre-therapy period comprising the temporal period before a said therapeutic intervention;
a concurrent-therapy period comprising the temporal period concurrent with a said therapeutic intervention;
a post-therapy period comprising the temporal period after a said therapeutic intervention;
said pre-therapeutic neurostimulation is delivered in the temporal period from 20 minutes to 0.001 second prior to a said therapeutic intervention to prepare the neurological environment of a human being for a said therapeutic intervention;
said concurrent neurostimulation is delivered concurrently, during the time period concurrent with a said therapeutic intervention to enhance the effectiveness of a said therapeutic intervention;
said post-therapeutic neurostimulation is delivered in the time period after a said therapeutic intervention to enhance recovery from a said therapeutic intervention or maximize the effect of a said therapeutic intervention.

13. The method of claim 11, wherein said neurostimulation is commenced in relation to a said at least one targeted exteroception marker selected from a group of targeted exteroception markers that includes a time of day, the presence of one or more persons, a situation, an interpersonal interaction, a behavioral event, an environmental event, an activity of daily living a life experience, and the like.

14. The method of claim 11, wherein said neurostimulation is commenced in relation to a said at least one targeted interoception marker selected from a group of targeted interoception markers that includes a mood, a perception of stress, a psychological experience, a bodily experience, a bodily sensation, breathing, awareness of bodily processes, nociception, and the like.

15. The method of claims 11 wherein biofeedback monitoring means are employed to monitor the biological signals of a person for the occurrence of a said at least one biofeedback marker;
said neurostimulation is conditional in relation a said at least one targeted biofeedback marker selected from a group of targeted biofeedback markers that includes:
at least one galvanic skin response (GSR) or skin conductance marker;
at least one electromyography marker indicating muscle tension or muscle activation;
at least one skin temperature marker obtained from a finger-applied temperature sensor;
at least one cardiologic activity marker such as particularly markers of heart rate, heart rate variability, normal sinus rhythm, respiratory sinus arrhythmia (RSA), and pathological arrhythmias;
at least one respiration marker, such as respiratory rate, expiration, inspiration, oxygenation, and the like;
at least one neurological marker, such as brainwave frequency, amplitude and dominance;
an index of autonomic nervous system activity used as a marker, includes heart rate variability frequency domains, low frequency and very low frequency;
said neurostimulation conditionality invokes changes in neurostimulation selected from a group that includes commencement of stimulation, cessation of stimulation, re-parameterization of stimulation and the like.

16. The method of claim 15, wherein said monitored biofeedback activity includes cardio-respiratory activity, neurological activity, skin conductance, electromyography, peripheral skin temperature, brainwave activity and the like;
said monitoring of said cardio-respiratory activity is accomplished using photoplethysmography;
said photoplethysmography is further configured to monitor at least one index of autonomic nervous system activity;
at least one algorithm is used to selectively control stimulation parameters according to algorithmic determinants and said monitored biofeedback activity;
said at least at least one algorithm incorporated as a neurostimulation controller means uses said monitored biofeedback to determine and adjust at least one stimulation parameter belonging to a group that includes energy frequency, energy intensity, stimulation time duration, energy pulse width, energy waveform, stimulation duty cycle, stimulation periodicity, total energy delivered and the like.

17. The method of claim 15 wherein at least one healthcare provider trains a person or group in the self-administer of said method of neurostimulation, said training comprising/including:
education regarding the identification and selection of exteroception marker targets relative to neurostimulation;
education regarding the identification and selection of interoception marker targets relative to neurostimulation;
education regarding the identification and selection of nociception marker targets relative to neurostimulation;
instruction in at least one beneficial purpose of using the said neurostimulation method;
instruction in decision-making regarding makers and indications for beneficially using said neurostimulation method;
training in the use of a said neurostimulation system, including parameterization, the application of a said at least one energy emitter to a said at least one cranial nerve target in the said auricular nerve field;
repeated supervised practice in recognizing said exteroception, interoception and nociception markers personally relevant to the subject individual;
training in decision-making about parameterizing and timing said neurostimulation
relative to any said personally relevant markers of exteroception, interoception and nociception;
repeated supervised practice using said neurostimulation wherein said practice includes preparing the skin, applying a said least one energy-emitter, and selecting stimulation parameters or an at least one pre-parameterized stimulation program.

18. The method of claim 17, wherein training in said self-administration of neurostimulation further includes the use biofeedback means to provide marker and determination guidance, further comprises;
education about the anatomy and function of the autonomic nervous system;
education about the bodily processes and biological signals sensed and monitored by biofeedback means;
education regarding the identification and selection of biofeedback marker targets relative to neurostimulation;
training in the selection and use of biofeedback monitoring means relative to neurostimulation;
supervised practice in setting up biofeedback means, and in reading and interpreting data obtained therefrom;
supervised practice in incorporating said data obtained from said biofeedback means into decision-making regarding the said timing and parameterization of said neurostimulation.

19. An improved, anatomically individualized, contour conforming auricular neurostimulation apparatus to apply energy stimulation to at least one of the cranial nerves accessible in the auricular nerve field, the apparatus comprising:
at least one conformal coupling means comprising a conformal plastic composition having at least one energy emitter means;
said at least one conformal coupling means couples said at least one energy emitter means to the ventral-ventrolateral surfaces of at least one auricle of a human being;
said at least one energy emitter means coupled to said at least one auricle of a human being delivers energy stimulation to said at least one of the cranial nerves accessible in the auricular nerve field;
said at least one of the cranial nerves accessible in the auricular nerve field is selected from a group that includes the auricular branch of the vagus nerve, the auriculotemporal nerve, the lesser occipital nerve and the great auricular nerve also known as the greater auricular nerve;
said at least one energy emitter means is comprised with said at least one conformal plastic composition to correspond anatomically and spatially with auricular anatomical landmarks associated with said at least one of the cranial nerves accessible in the ventral-ventrolateral auricular nerve field;
said anatomical landmarks include the cymba conchae, cavum concha, the tragus, the lobule also known as the ear lobe, the fossa triangularis, the superior crus of the antihelix, the inferior crus of the anti-helix, the helix, the scapha and the like;
said at least one conformal plastic composition is comprised of a group of plastic materials that includes thermo-settable plastics, photonic-settable plastics and mechanically-settable plastics;
said at least one energy emitter means is selected from a group of energy emitters means that includes emitters of electrical energy, optical emitters of electromagnetic energy, and emitters of vibrational energy;
said at least one energy emitter means is selected from a group that includes metallic electrodes, conductive metal, optical emitters, haptic emitters, graphene emitters, conductive filaments, conductive ink, conductive plating and the like;
electrically conductive pathways on said conforming plastic composition are composed of conductive compositions selected from a group that includes screen-printed carbon ink, 3-D printed conductive filaments, metallic tracing, screen printed conductive metallic inks and the like.

20. The contour conforming auricular neurostimulation apparatus of claim 19, further comprising:
a second said conformal coupling means comprising a conformal plastic composition having at least one energy emitter means;
said second conformal coupling means couples said at least one energy emitter means to the dorsal-dorsolateral surfaces of at least one auricle of a human being;
said at least one energy emitter means is comprised with said at least one conformal plastic composition to anatomically and spatially correspond with auricular anatomical landmarks associated with at least one of the cranial nerves accessible in the dorsal-dorsolateral auricular nerve field;
said at least one of the cranial nerves accessible in the auricular nerve field is selected from a group that includes the auricular branch of the vagus nerve, the auriculotemporal nerve, the lesser occipital nerve and the great auricular nerve also known as the greater auricular nerve;
said anatomical landmarks include the posterior crus, the eminence of the triangular, also known as the eminence of the fossa triangularis, the eminence of the cymba conchae, the eminence of the cavum conchae, the antihelical fossa, the posterior antihelix, the posterior lobule, and the like;
said at least one energy emitter comprised said second conformal coupling means is configured for electronic communication with a neurostimulation device.
